(19) United States
(12) Patent Application Publication
(10) Pub. No.: US 2010/0240612 A1
(43) Pub. Date: Sep. 23, 2010

(54) Prenylated Bisphosphonates as Anti-tuberculosis Agents

(75) Inventors: David F. Wiemer, Iowa City, IA (US); Rocky J. Barney, Iowa City, IA (US); Raymond J. Hohl, Iowa City, IA (US)

Correspondence Address: VIKSNINS HARRIS & PADYS PLLP, P.O. BOX 111098, ST. PAUL, MN, 55111-1098, US

(73) Assignee: University of Iowa Research Foundation, Iowa City, IA (US)

(21) Appl. No.: 12/729,111

(22) Filed: Mar. 22, 2010

Related U.S. Application Data
(60) Provisional application No. 61/162,145, filed on Mar. 20, 2009.

(51) Int. Cl. A61K 31/675; A61K 31/663; C07F 9/38; C12N 9/99; A61P 31/06; A61P 31/04

(52) U.S. Cl. 514/80; 514/107; 562/21; 548/414; 435/184

(57) ABSTRACT

The invention provides methods to treat a mycobacterium infection and methods to inhibit mycobacterial polyprenyl pyrophosphate synthesis with a compound of formula I. The invention also provides novel compounds of formula I as well as salts and prodrugs thereof.

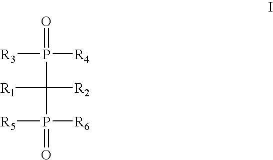

### RELATED APPLICATION

This patent document claims the benefit of priority of U.S. application Ser. No. 61/162,145, filed Mar. 20, 2009, which application is herein incorporated by reference.

### BACKGROUND

Mycobacterium infections continue to be a significant health issue throughout the world. Tuberculosis is a common and deadly infectious disease that is caused by mycobacterium, particularly Mycobacterium tuberculosis.

There is currently a need for therapeutic methods that are useful for treating mycobacterium infections. There is also a particular need for therapeutic methods that are useful for treating tuberculosis by inhibiting mycobacterial polyprenyl pyrophosphate synthesis.

### SUMMARY OF CERTAIN EMBODIMENTS OF THE INVENTION

The present invention provides methods to treat a mycobacterium infection (e.g. tuberculosis) in a mammal (e.g. a human) by administering compounds that inhibit mycobacterial polyprenyl pyrophosphate synthesis.

Accordingly the invention provides a method to treat a mycobacterium infection (e.g. tuberculosis) in a mammal (e.g. a human) comprising administering a compound of formula I:

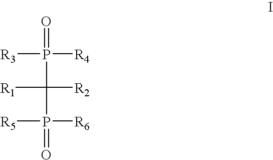

wherein:

R₁ is a saturated or unsaturated (C₅-C₂₀)alkyl chain that optionally comprises one or more aryl or heteroaryl rings in the chain wherein (C₅-C₂₀)alkyl is optionally substituted with one or more halo, cyano, nitro, carboxy, trifluoromethyl, trifluoromethoxy, NRₘRₙ, or S(O)₂NRₚR_{q} and wherein any aryl or heteroaryl is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d};

R₂ is H, halo, OH, trifluoromethyl, —ORₑ, NR_{f}R_{g} or a saturated or unsaturated (C₁-C₆)alkyl wherein (C₁-C₆)alkyl is optionally substituted with one or more halo;

each R₃, R₄, R₅, and R₆ is independently OH or (C₁-C₆)alkoxy;

each Rₐ and R_{b} is independently H, (C₁-C₆)alkyl, or aryl; or Rₐ and R_{b} together with the nitrogen to which they are attached form a pyrrolidino, piperidino, morpholino, or thiomorpholino ring;

each R_{c} and R_{d} is independently H, (C₁-C₆)alkyl, or aryl; or R_{c} and R_{d} together with the nitrogen to which they are attached form a pyrrolidino, piperidino, morpholino, or thiomorpholino ring;

each Rₑ is independently (C₁-C₆)alkyl or aryl;

each R_{f} and R_{g} is independently H, (C₁-C₆)alkyl, or aryl; or R_{f} and R_{g} together with the nitrogen to which they are attached form a pyrrolidino, piperidino, morpholino, or thiomorpholino ring;

each Rₚ and Rₙ is independently H, (C₁-C₆)alkyl, or aryl; or Rₘ and Rₙ together with the nitrogen to which they are attached form a pyrrolidino, piperidino, morpholino, or thiomorpholino ring;

each Rₚ and R_{q} is independently H, (C₁-C₆)alkyl, or aryl; or Rₚ and R_{q} together with the nitrogen to which they are attached form a pyrrolidino, piperidino, morpholino, or thiomorpholino ring; and

wherein any aryl of Rₐ, R_{b}, R_{c}, R_{d}, Rₑ, R_{f}, R_{g}, Rₘ, Rₙ, Rₚ or R_{q} is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₛRₜ, or S(O)₂NRₛRₜ wherein each Rₛ and Rₜ is independently H or (C₁-C₆)alkyl;

or a pharmaceutically acceptable salt or prodrug thereof to the mammal.

The invention also provides a method for inhibiting the activity of a mycobacterial polyprenyl pyrophosphate synthase in vitro or in vivo comprising contacting the mycobacterial polyprenyl pyrophosphate synthase with an effective amount of a compound of formula I as described herein.

The invention also provides a compound of formula I as described herein or a pharmaceutically acceptable salt or prodrug for use in the prophylactic or therapeutic treatment of a mycobacterium infection (e.g. tuberculosis).

The invention also provides the use of a compound of formula I as described herein or a pharmaceutically acceptable salt or prodrug thereof for the manufacture of a medicament useful for treating a mycobacterium infection (e.g. tuberculosis) in a mammal (e.g. a human).

The invention also provides the use of a compound of formula I or a pharmaceutically acceptable salt or prodrug thereof as described herein for the manufacture of a medicament useful for inhibiting the activity of a mycobacterial polyprenyl pyrophosphate synthase in a mammal (e.g. a human).

The invention also provides novel compounds of formula I as described herein or salts or prodrugs thereof.

### BRIEF DESCRIPTION OF THE FIGURES

FIG. 1 illustrates the inhibition of mycobacterial polyprenyl synthase (RV2361c) by compound 13.

FIG. 2 illustrates the inhibition of mycobacterial polyprenyl synthase (RV2361c) by compound 23.

FIG. 3 illustrates the inhibition of bacterium M. Smegmatis by compound 24.

### DETAILED DESCRIPTION

The invention also provides novel synthetic intermediates and processes described herein.

The following definitions are used, unless otherwise described: halo is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to. Unsaturated (C₁-C₂₀)alkyl denotes a (C₂-C₂₀)alkyl with at least one unsaturated (i.e. double or triple) bond. Unsaturated (C₅-C₂₀)alkyl denotes a (C₅-C₂₀)alkyl with at least one unsaturated (i.e. double or triple) bond Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

As used herein, a saturated or unsaturated (C₅-C₂₀)alkyl chain that comprises one or more aryl or heteroaryl rings in the chain includes: 1) alkyl chains that have an aryl or hetereoaryl within the chain so as to have one portion of the alkyl chain attached to one atom of the aryl or heteroaryl and another portion of the alkyl chain attached to a different atom of the aryl or heteroaryl and 2) alkyl chains that are terminated with an aryl or heteroaryl.

In one embodiment of the invention, the saturated or unsaturated (C₅-C₂₀)alkyl chain that comprises one or more aryl or heteroaryl rings in the chain of R₁, includes the aryl or hetereoaryl within the chain so as to have one portion of the alkyl chain attached to one atom of the aryl or heteroaryl and another portion of the alkyl chain attached to a different atom of the aryl or heteroaryl.

The term “prodrug” is well understood in the art and includes compounds that are converted to pharmaceutically active compounds in vivo (e.g. in an animal such as a mammal). For example, see Remington's Pharmaceutical Sciences, 1980, vol. 16, Mack Publishing Company, Easton, Pa., 61 and 424. In particular, a number of groups suitable for preparing prodrug forms of phosphorous containing compounds (e.g. phosphonates) are known. For example, see Galmarini C M, et al., International Journal of Cancer, 2003, 107 (1), 149-154; Wagner, C. R., et al., Medicinal Research Reviews, 2000, 20, 417-51; McGuigan, C., et al., Antiviral Research, 1992, 17, 311-321; and Chapman, H., et al., Nucleosides, Nucleotides & Nucleic Acids, 2001, 20, 1085-1090. The invention includes phosphonate prodrug analogs prepared from suitable in vivo hydrolysable groups. In one specific embodiment the invention provides for phosphonate prodrugs of the compounds of formula I wherein the phosphonate is pivaloyloxymethyl (i.e. wherein one or more of R₃, R₄, R₅ or R₆ is —OCH₂OC(O)C(CH₃)₃).

It will be appreciated by those skilled in the art that compounds of the invention having a chiral center may exist in and be isolated in optically active and racemic forms. For example, it is possible for one or both phosphorous atoms in a compound of formula Ito be chiral centers. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine enzyme inhibitory activity using the standard tests that are well known in the art.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkanoyloxy can be formyloxy, acetoxy, propanoyloxy, butanoyloxy, isobutanoyloxy, pentanoyloxy, or hexanoyloxy; and aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for R₁ is an unsaturated (C₅-C₂₀)alkyl chain.

Another value for R₁ is a saturated or unsaturated (C₅-C₂₀)alkyl chain that comprises one or more aryl rings in the chain.

Another value for R₁ is a unsaturated (C₅-C₂₀)alkyl chain that comprises an aryl ring in the chain.

Another value for R₁ is a saturated or unsaturated (C₅-C₂₀)alkyl chain that comprises one or more heteroaryl rings in the chain.

Another value for R₁ is a unsaturated (C₅-C₂₀)alkyl chain that comprises a heteroaryl ring in the chain.

A specific value for R₁ is the formula,

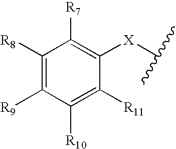

wherein:

one of R₇, R₈, R₉, R₁₀ and R₁₁ is Y and the others are each independently H, halo, cyano, nitro, carboxy, trifluoromethyl, trifluoromethoxy (C₁-C₆)alkyl, NRⱼRₖ, or S(O)₂NRⱼRₖ wherein Rⱼ and Rₖ are each H or (C₁-C₆)alkyl;

Y is a saturated or unsaturated (C₁-C₂₀)alkyl;

X is (CRₕRᵢ)ₙ wherein n is 0, 1, 2, 3, 4, or 5 and for each CRₕRᵢ; Rₕ and Rᵢ are each independently H or (C₁-C₃)alkyl; and

provided that the sum of the carbons of X and Y is 5 to 20.

A specific group of compounds of formula I are compounds wherein Rₕ and Rᵢ are each H.

A specific value for n is 1.

A specific value for R_{g} is Y.

A specific value for R₉ is Y.

A specific value for Y is a saturated or unsaturated (C₅-C₂₀)alkyl.

Another value for Y is 3-methyl-2-buten-1-yl.

A specific value for R₁ is

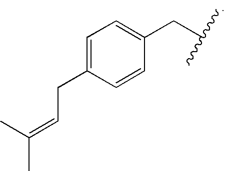

Another value for R₁ is

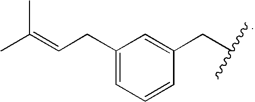

Another value for R₁ is

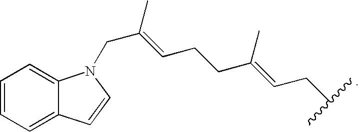

Another value for R₁ is

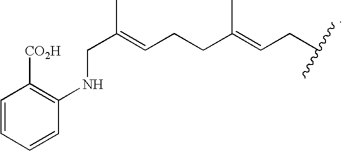

A specific value for R₂ is saturated or unsaturated (C₁-C₆)alkyl, OH or H.

Another value for R₂ is saturated or unsaturated (C₁-C₄)alkyl, OH or H.

Another value for R₂ is saturated or unsaturated (C₁-C₃)alkyl, OH or H.

Another value for R₂ is OH or H.

Another value for R₂ is H.

In one embodiment a compound of formula I is

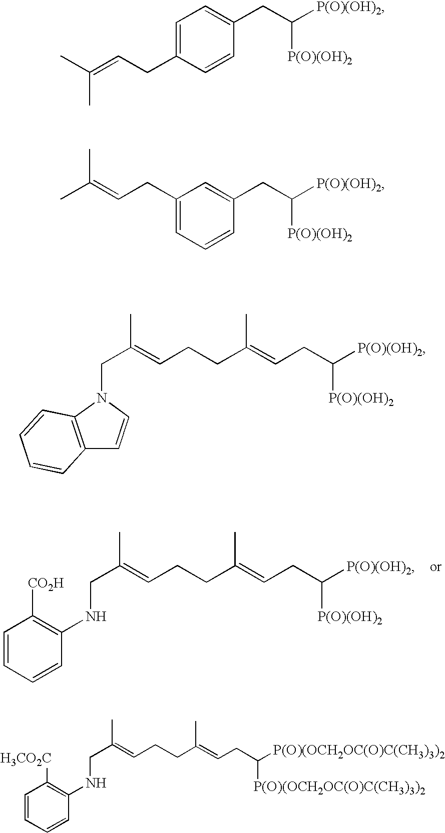

or a salt or prodrug thereof.

In another embodiment a compound of formula I is

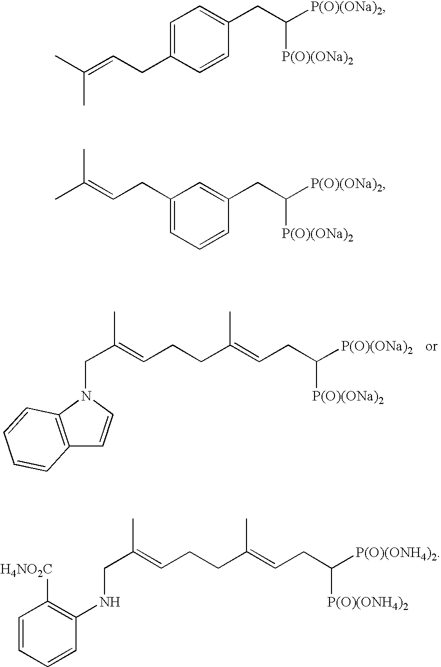

A specific group of compounds of formula I are compounds wherein R₃, R₄, R₅ and R₆ are each OH.

The invention provides novel compounds disclosed herein. For example, the invention provides novel compounds of formula I wherein R₁ is a saturated or unsaturated (C₅-C₂₀)alkyl chain that comprises one or more heteroaryl rings and optionally comprises one or more aryl rings in the chain wherein (C₅-C₂₀)alkyl is optionally substituted with one or more halo, cyano, nitro, carboxy, trifluoromethyl, trifluoromethoxy, NRₘRₙ, or S(O)₂NRₚR_{q} and wherein any aryl or heteroaryl is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d};

provided that when R₁ is a saturated or unsaturated (C₅-C₂₀)alkyl chain that comprises one pyridine ring, the pyridine ring is not linked to the alkyl chain of R₁ through the pyridine nitrogen; and

provided that the compound is not

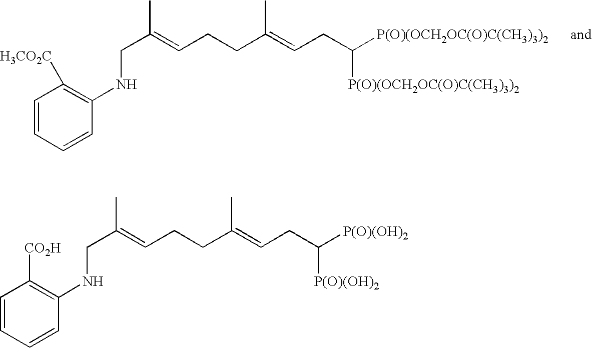

In one embodiment the invention excludes compounds of formula I wherein R₁ is:

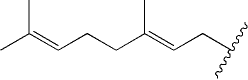

substituted with NRₘRₙ.

In another embodiment the invention excludes compounds of formula I wherein R₁ is:

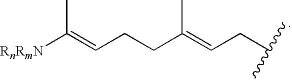

In another embodiment the invention excludes compounds of formula I wherein R₁ is:

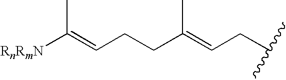

and wherein Rₙ is H or (C₁-C₆)alkyl and Rₘ is phenyl substituted with carboxy.

The invention also provides novel compounds of formula I wherein R₁ is —(C₅-C₂₀)alkyl-Z¹ wherein (C₅-C₂₀)alkyl is saturated or unsaturated and is optionally substituted with one or more halo, cyano, nitro, carboxy, trifluoromethyl, trifluoromethoxy, NRₘRₙ, or S(O)₂NRₚR_{q}; and wherein Z¹ is heteroaryl optionally substituted with one or more (e.g. 1, 2, 3 or 4) (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d}.

A specific value for Z¹ is furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

Another specific value for Z¹ is furyl, imidazolyl, triazolyl, triazinyl, oxazoyl, isoxazoyl, thiazolyl, isothiazoyl, pyrazolyl, pyrrolyl, pyrazinyl, tetrazolyl, thienyl, pyrimidinyl (or its N-oxide), indolyl, isoquinolyl (or its N-oxide) or quinolyl (or its N-oxide).

Another specific value for Z¹ is furyl, triazolyl, triazinyl, oxazoyl, isoxazoyl, thiazolyl, isothiazoyl, pyrazolyl, pyrrolyl, pyrazinyl, tetrazolyl, thienyl, pyrimidinyl (or its N-oxide), indolyl, isoquinolyl (or its N-oxide) or quinolyl (or its N-oxide).

Another specific value for Z¹ is indolyl.

Representative compounds used in the methods of the invention can be prepared as illustrated in Schemes 1-2.

Representative compounds of the invention can be prepared as illustrated in Schemes 1-2 wherein the phenyl group has been replaced by a heteroaryl group.

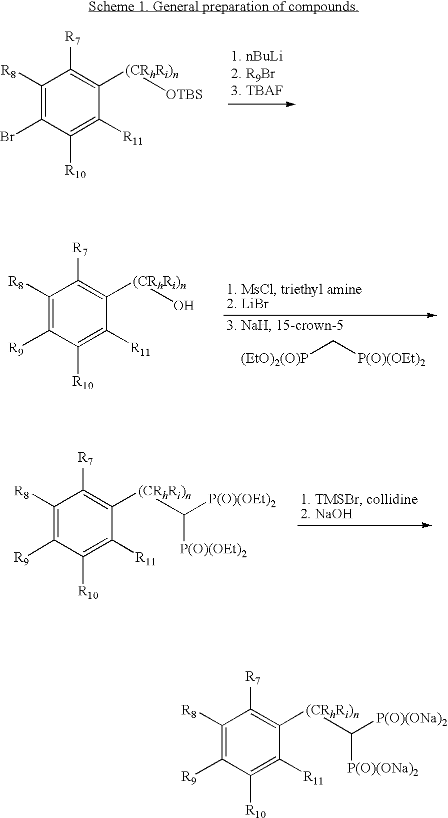

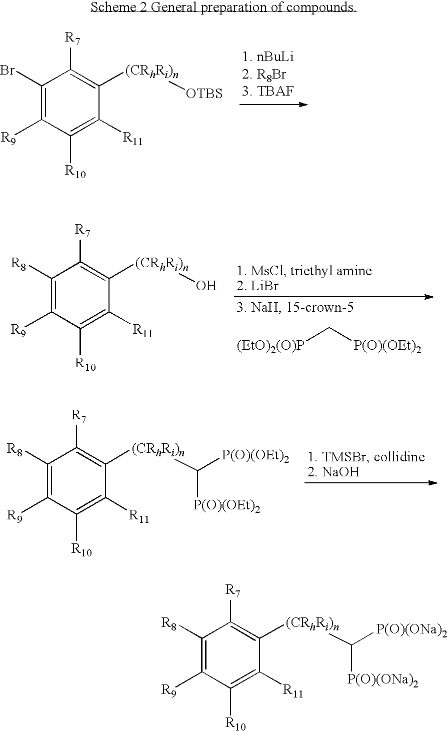

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Certain embodiments of the present invention provide methods of inhibiting the activity of a mycobacterial polyprenyl pyrophosphate synthase in vivo or in vitro comprising contacting the mycobacterial polyprenyl pyrophosphate synthase with an effective amount of a compound of formula I as described herein. The methods also include inhibiting a mycobacterial polyprenyl pyrophosphate synthase with an effective amount of a compound of formula I in an animal (e.g. a mammal such as a human). The invention also provides methods of inhibiting the activity of a mycobacterial polyprenyl pyrophosphate synthase with a compound of formula I in a sample wherein the sample includes but is not limited to an aqueous solution (e.g. sputum), a tissue, a biopsy or a cell. The invention also provides methods of using the compounds of formula I as probes for investigating the activity of a mycobacterial polyprenyl pyrophosphate synthase in vitro or in vivo.

The ability of a compound of the invention to act as an inhibitor of mycobacterial polyprenyl pyrophosphate infections may be determined using pharmacological models which are well known in the art and as described in Test A below. It is known that mycobacterial polyprenyl pyrophosphate synthase is essential for the viability of Mycobacterium tuberculosis (Crick, D. C., Schulbach, M. C., Zink, E. E., et al., Journal of Bacteriology, 2000, 182 (20), 5771-5778; Sassetti, C. M., Boyd, D. H., Rubin, E. J., Mol. Microbiol., 2003, 48(1), 77-84). Therefore, compounds that have inhibitory activity against mycobacterial polyprenyl pyrophosphate synthase may be useful as therapeutic agents to treat tuberculosis.

The ability of a compound of the invention to inhibit the growth mycobacterium M. Smegmatis may be determined using pharmacological models which are well known in the art and as described in Test B below. Therefore, compounds that inhibit the growth of mycobacterium M. Smegmatis may be useful as therapeutic agents to treat mycobacterium infections including tuberculosis.

### Test A: Inhibition of Mycobacterial Polyprenyl Synthase (RV2361c)

A plasmid containing N-terminal His tagged decaprenyl diphosphate synthase (Rv2361c) was transformed into E. coli DE3 BL21 star (Invitrogen). Bacteria were then grown to log phase and expression was induced by addition of 0.1 mM IPTG overnight at room temperature. Cells were then pelleted by centrifugation and then resuspended in 50 mM Tris pH 8 containing 300 mM NaCl, 10 mM imidazole, and 1 mM phenylmethanesulphonylfluoride (PMSF). Cells were lysed using 1 mg/ml lysosome for 30 minutes at room temperature. Lysate was loaded onto a His Select (Sigma) nickel affinity resin column, washed, and eluted with 250 mM imidazole according to the manufacturer instructions, except 0.1% Triton-X-100 (TX100) was added to final washes and included in the elution buffer.

Enzyme assays were typically performed in 20 μl reactions containing 50 mM Tris pH 7.9 buffer, 1 mM MgCl₂, 0.15% Triton-X-100, 1 mM DTT, 30 ng recombinant enzyme, and unless otherwise noted 100 μM FPP and 30 μM ¹⁴C IPP. Compounds were added prior to substrate addition and allowed to preincubate for 10 min at 37° C., after which substrates were added and reactions were allowed to proceed for 10 min at 37° C. Next, 300 μl of butanol saturated water was added followed by 1 ml of water saturated butanol. The butanol layer containing the product was extracted, washed, and then radioactivity was quantitated using a liquid scintillation counter.

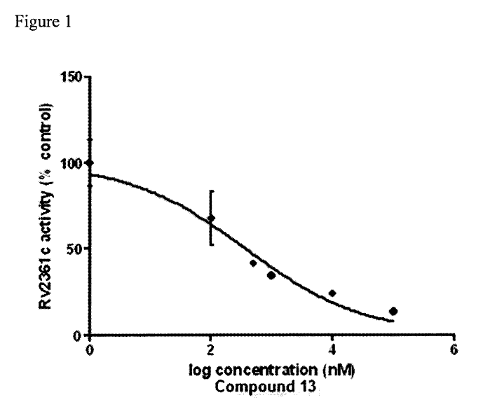

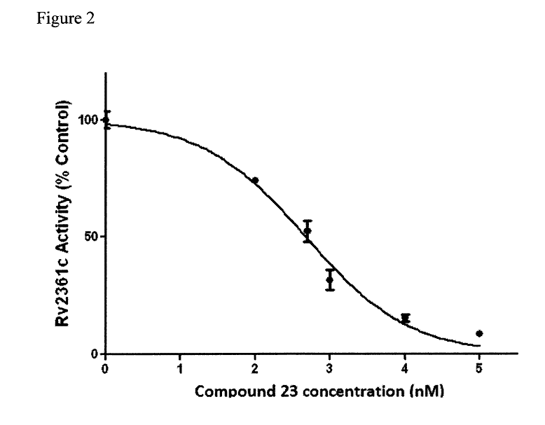

Compounds of formula I were found to have activity against mycobacterial polyprenyl pyrophosphate synthase. FIG. 1 shows the inhibition of purified mycobacterial polyprenyl synthase (RV2361c) by compound 13 while FIG. 2 shows the inhibition of RV2361c by compound 23.

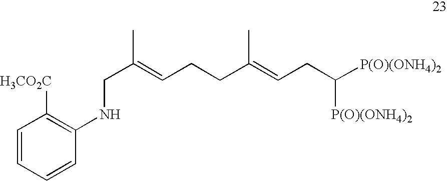

### Test B: Inhibition of Growth of the Bacterium M. Smegmatis

Mycobacterium Smegmatis (ATCC 607) was obtained from American Type Culture Collection and cultured in 7H9 broth (Sigma) containing ADC (Sigma), 0.5% glycerol, and 0.05% Tween-80. Bacteria were grown to log phase and then diluted, aliquoted, and grown in the presence of indicated drugs for 2 days. Growth was measured as a function of optical absorbance at 540 nM using a spectrophotometer. Alternatively, aliquots of liquid cultures were spread onto agar containing plates, which were then grown for 2 days, after which the number of colony forming units (CFU) were counted and normalized per mL.

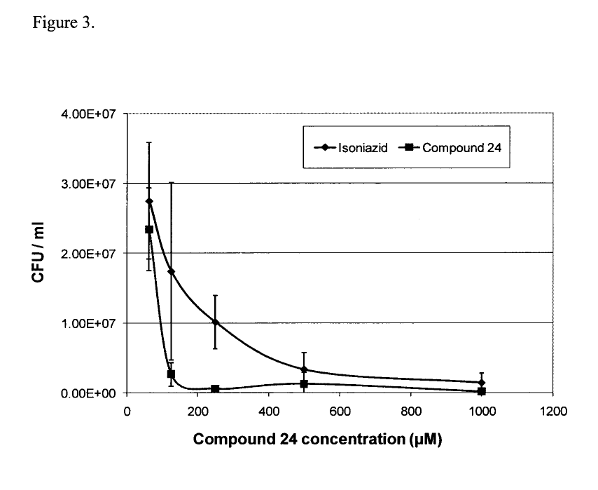

FIG. 3 shows the inhibition of growth of the bacterium M. Smegmatis by compound 24 versus the compound Isoniazid (isonicotinylhydrazine).

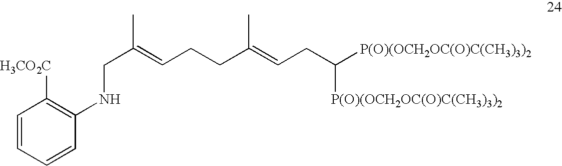

The invention will now be illustrated by the following non-limiting Examples.

### General

Compounds were identified using ¹H NMR, ¹³C NMR, ³¹P NMR or comparison to authentic sample where applicable. Glassware was flame-dried prior to use. Reactions were carried out with stirring under a positive argon atmosphere unless otherwise indicated. All NMR data were collected at 300 MHz in CDCl₃ unless otherwise noted.

### Example 1

### Synthesis of Compound 13

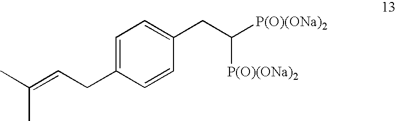

p-Prenyl-phenethylidene bisphosphonic acid tetra sodium salt (13). A stirred solution of compound 12 (0.775 g, 1.74 mmol, 1.00 eq) in CH₂Cl₂ was cooled to 0° C. in an icebath. 2,4,6-collidine (1.16 mL, 8/5 mmol, 5.03 eq) was added followed by slow addition of TMSBr (1.13 mL, 8.74 mmol, 5.02 eq) and allowed to stir overnight. Toluene was added and removed in vacuo. This process was repeated three times. After drying of the crude material, the residue was treated with NaOH solution (1.73M, 5 mL, 8.65 mmol, 4.97 eq) and allowed to stir at room temperature overnight. Acetone was added to the solution and the resulting material was allowed to cool at 3° C. for 72 hr. The solution was filtered, the solid was washed with several portions of cold acetone and dried in vacuo. In D₂0 ¹H NMR δ 1.70 (3H, bs), 1.71 (3H, bs), 2.29 (1H, tt, J_{HP}=21.9 Hz, J=5.8 Hz), 3.08 (2H, dt, Jₜ=16.3 Hz, J_{d}=6.9 Hz), 3.31 (2H, d, J=7.5 Hz), 5.35-5.42 (1H, m), 7.17 (2H, d, J=7.8 Hz), 7.29 (2H, d, J=8.1 Hz) ³¹PNMR (proton decoupled) δ 20.24.

Compound 12 was prepared as follows.

a) p-Bromobenzyl-t-butyldimethylsilyl ether (10). Under an argon atmosphere, imidazole (4.39 g, 64 mmol, 2.5 eq.) was added with stirring to a solution of 4-bromobenzyl alcohol (4.73 g, 25.7 mmol, 1.0 eq.) in CH₂Cl₂. The solution was cooled to 0° C., followed by the addition of TBSCl (4.70 g, 31.2 mmol, 1.2 eq.). The reaction mixture was allowed to stir overnight. The solution was quenched with H₂O, extracted with CH₂Cl₂, dried (MgSO₄), and concentrated. Purification using flash chromatography through a short plug of silica gel (15% EtOAc in hexanes) afforded the TBS protected alcohol compound 10 in 96% yield (7.40 g). 1H NMR δ 0.07 (6H, s), 0.92 (9H, s), 4.67 (2H, S), 7.17 (2H, d J=8.7 Hz), 7.42 (2H, d, J=8.4 Hz) ¹³CNMR δ 140.4, 131.2 (2C), 127.7 (2C), 120.5, 64.3, 25.9 (3C), 18.5, −5.3 (2C).

b) p-Prenylbenzyl alcohol (11). A stirred solution of compound (10) (6.61 g, 21.9 mmol, 1.0 eq.) in THE was cooled to −78° C. in a dry-ice/acetone bath. Once cooling was complete, tiBuLi (11.5 mL, 2.1M in THF, 1.1 eq.) was added slowly via syringe, and the solution was allowed to stir for fifteen minutes. Prenyl bromide (4.26 g, 28.6 mmol, 1.3 eq.) was added dropwise via syringe. The solution was held at −78° C. for two hours, and then allowed to gradually warm to room temperature and stir overnight. The resulting mixture was quenched with H₂O, extracted with diethyl ether, dried (MgSO₄), and concentrated in vacuo. The crude mixture was moved forward without additional purification. A stirred solution of the crude material in THF was cooled to 0° C. in an ice-bath and a 1M solution of TBAF in THF (27.1 mL, 27.1 mmol, 1.0 eq.) was added dropwise to the reaction vessel. The reaction was allowed to stir for 4 hours, at which point the solution was diluted with diethyl ether and H₂O was added. The resulting mixture was extracted with diethyl ether, dried (MgSO₄), and concentrated in vacuo. Purification using flash column chromatography (10% EtOAc in hexanes) afforded compound 11 in 77% yield (2.99 g) over two steps. ¹, HNMR δ 1.70 (3H, s), 1.73 (3H, s), 2.78 (1H, bs), 3.31 (2H, d, J=7.5 Hz), 4.51 (2H, s), 5.25-5.33 (1H, m), 7.12 (2H, d, J=8.4 Hz), 7.20 (2H, d, J=8.1 Hz) ¹³C NMR δ 141.0, 138.2, 132.4, 128.3 (2C), 127.1 (2C), 123.0, 64.7, 33.9, 25.6, 17, 7 HRMS (ESI, m/z) calcd for (M)⁺C₁₂H₁₆0:176.1201. Found 176.1199.

c) p-Prenyl-phenethylidene bisphosphonic acid tetraethyl ester (12). A stirred solution of compound (11) (4.46 g, 25.3 mmol, 1.0 eq) in CH₂Cl₂ was cooled to 0° C. in an ice bath. To the cooled solution was added dry triethylamine (4.58 mL, 40.0 mmol, 1.6 eq) followed by dropwise addition of MsCl (2.36 mL, 30.5 mmol, 1.2 eq). The solution was allowed to stir for 30 minutes. LiBr (5.55 g, 63.9 mmol, 2.5 eq) was placed into a dry flask under argon atmosphere and dissolved in dry THF. This was then transferred via syringe into the reaction vessel. The reaction mixture was allowed to stir for 1.5 hours at which point the solution was quenched by addition of H₂O followed by addition of saturated NaCl solution. The resulting solution was extracted with CH₂Cl₂, dried (Na₂SO₄) and concentrated in vacuo to provide the crude bromide.

To a stirred solution of NaH (60% in oil, 0.932 g, 23.3 mmol, 1.0 eq) in THF was added 15-crown-5 (0.51 mL, 2.58 mmol, 0.1 eq). The solution was cooled to 0° C. in an ice bath. After complete cooling of the solution, tetraethyl methylenebisphosphonate (7.38 g, 25.6 mmol, 1.1 eq) was added slowly via syringe and allowed to stir for 30 minutes to facilitate complete formation of the anion. Next, a solution of the crude bromide in THF was added slowly, via syringe, to the reaction vessel. The mixture was immediately removed from the ice bath and allowed to stir overnight. The solution was filtered through a bed of fluorasil and concentrated in vacuo. Purification was achieved using flash column chromatography (gradient 6%-8% EtOH in hexanes) to afford the target bisphosphonate compound 12 in 61% (6.89 g) and a by product compound (12b) in 2% (0.311 g) yield over two steps. Compound 12 ¹H NMR δ 7.11 (2H, dd, Jₒ=8.3, Jₘ=2.1 Hz), 7.01 (2H, dd, Jₒ=7.8, Jₘ=2.1 Hz), 5.23-5.16 (1H, m), 4.10-3.94 (8H, m), 3.22 (2H, d, J=7.2 Hz), 3.09 (2H, td, Jₜ=6.3, J_{d}=1.8 Hz), 2.68-2.45 (1H, m), 1.65 (3H, s), 1.63 (3H, s), 1.19 (12H, td, Jₜ=7.2, Jₚ=2.7 Hz) ¹³CNMR δ 139.8, 136.7 (1C, t, Jₚ=7.4 Hz), 132.1, 128.7 (2C), 127.9 (2C), 123.1, 62.4-62.1 (4C, m), 38.9, 33.7, 30.6, 25.5, 17.6, 16.1 (4C, d, Jₚ=7.2 Hz), ³¹P NMR δ 23.6; di-alkylated bisphosphonate Compound 12b, ¹H NMR δ 7.35 (4H, d, Jₒ=7.8 Hz), 7.05 (4H, d, Jₒ=7.8 Hz), 5.33-5.25 (2H, m), 4.04-3.92 (8H, m), 3.36-3.25 (8H, m), 1.72 (6H, s), 1.70 (6H, s), 1.13 (12H, t, Jₜ=6.9 Hz), ¹³C NMR δ 139.8 (2C), 133. 2C, t, Jₚ=6.5 Hz), 131.8 (2C), 131.5 (4C), 127.0 (4C), 123.3 (2C), 62.0-61.8 (4C, m), 48.7 (1C, t, Jₚ=130.2), 37.1 (2C, 4.8 Hz), 33.8 (2C), 25.5 (2C) 17.5 (2C), 15.8 (4C, t, Jₚ=3.5 Hz), ³¹PNMR δ 24.5. HRMS (ESI, m/z) calcd for (M)⁺C₃₃H₅₀O₆P₂:604.3083. Found 604.3086.

### Example 2

### Synthesis of Compound 17

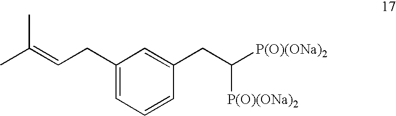

m-Prenyl-phenethylidene bisphosphonic acid tetra sodium salt (17). A stirred solution of compound 16 (0.767 g, 1.72 mmol, 1.00 eq) in CH₂Cl₂ was cooled to 0° C. M an icebath. 2,4,6-collidine (1.14 mL, 8.60 mmol, 5.00 eq) was added followed by slow addition of TMSBr (1.11 mL, 8.58 mmol, 4.99 eq) and allowed to stir overnight. Toluene was added and removed in vacuo. This process was repeated three times. After drying of the crude material, the residue was treated with NaOH solution (1.73M, 5 mL, 8.65 mmol, 5.03 eq) and allowed to stir at room temperature overnight. Acetone was added to the solution and the resulting material was allowed to cool at 3° C. for 72 hr. The solution was filtered; the solid was washed with several portions of cold acetone and dried in vacuo. In D₂0 ¹HNMR δ 1.72 (3H, s), 1.73 (3H, s), 2.19 (1H, tt, J_{PH}=21.0 Hz, J=6.3 Hz), 3.08 (2H, dt, J_{d}=6.3 Hz, Jₜ=15.3 Hz), 3.35 (2H, d, J=7.5 Hz), 5.38-5.44 (1H, m), 7.06-7.10 (1H, m), 7.22-7.31 (3H, m). ³¹P NMR (proton decoupled) δ 20.04.

Compound 16, was prepared as follows.

a) m-Prenylbenzy t-butyldimethylsilyl ether (14). To a solution of 3-bromobenzyl alcohol (26.7 mmol, 1.00 eq) in CH₂Cl₂ at 0° C. was added imidazole (130.6 mmol, 4.90 eq) followed by TBSCI (34.7 mmol, 1.30 eq). The reaction mixture was allowed to warm to room temperature and left to stir overnight. The solution was quenched by addition of H₂O, extracted with CH₂Cl₂, dried (MgSO₄), and concentrated in vacuo. Final purification using flash chromatography (8% EtOAc in hexanes) afforded the TBS protected alcohol in 90% yield (7.26 g). Both the ¹H NMR and ¹³C NMR data correlated to literature values. (Ref: Matsuda, Kazuhiko; Hamada, Masayuki; Nishimura, Keiichiro; Fujita, Toshio. Quantitative structure-activity studies of pyrethroids. 17. Physicochemical substituent effects of substituted benzyl esters of kadethric acid on symptomatic and neurophysiological activities. Pesticide Biochemistry and Physiology (1989), 35(3), 300-14.)

b) m-Prenylbenzyl alcohol (15). A stirred solution of compound 14 (5.89 g, 19.5 mmol, 1.0 eq) in THF was cooled to −78° C. in a dry-ice/acetone bath. Once cooling was complete, a 2.1 M solution of nBuLi in hexanes (10.2 ml, 21.5 mmol, 1.1 eq.) was added slowly via syringe, and the solution was allowed to stir for 15 minutes. Afterward, prenyl bromide (3.82 g, 25.6 mmol, 1.31 eq.) was added dropwise via syringe and the solution was held at −78° C. for two hours, then allowed to warm gradually to room temperature, and stirred overnight. The resulting mixture was quenched by addition of H₂O, extracted with diethyl ether, dried with MgSO₄, and concentrated in vacuo. The crude product was carried forward without additional purification. The crude product was dissolved in THF to make a solution approximately 2M in concentration. The solution was cooled to 0° C. and a 1M solution of TBAF in THF (23.3 mmol, 1.2 eq) was added dropwise to the reaction vessel. The reaction was allowed to stir for 4 hours, at which point the reaction was quenched by the addition of H₂O. The resulting mixture was extracted with diethyl ether, dried (MgSO₄), and concentrated in vacuo. Final purification by flash chromatography (10% EtOAc in hexanes) gave the target compound in 72% yield (2.48 g) over two steps. ¹HNMR δ 1.70 (3H, s), 1.73 (3H, s), 2.57 (1H, bs), 3.31 (2H, d, 7.2 Hz), 4.54 (2H, s), 5.26-5.44 (1H, m), 7.06-7.13 (3H, m), 7.20-7.26 (1H, m) ¹³C NMR δ 142.0, 140.9, 132.4, 128.5, 127.4, 126.9, 124.3, 122.9, 65.0, 34.2, 25.6, 17.7 HRMS (ESL m/z) calcd for (M) C₁₂H₁₆0:176.1201. Found 176.1204.

c) m-Prenyl-phenethylidene bisphosphonic acid tetraethyl ester (16). A stirred solution of compound 15 (2.00 g, 11.4 mmol, 1.0 eq) in CH₂Cl₂ was cooled to 0° C. in an ice bath. To the cooled solution was added dry triethylamine (2.01 mL, 14.5 mmol, 1.3 eq) followed by dropwise addition of MsCl (1.05 mL, 13.6 mmol, 1.2 eq). The solution was allowed to stir for 30 minutes. LiBr (2.52 g, 29.0 mmol, 2.6 eq) was placed into a dry flask under argon atmosphere and dissolved in dry THF. This was then transferred via syringe into the reaction vessel. The reaction mixture was allowed to stir for 1.5 hours at which point the solution was quenched by addition of H₂O followed by addition of saturated NaCl solution. The resulting solution was extracted with CH₂Cl₂, dried (Na₂SO₄), filtered and concentrated in vacuo. No additional purification was performed as the crude bromide was utilized in the next reaction.

To a stirred solution of NaH (60% in oil, 0.437 g, 10.9 mmol, 1.0 eq) in THF was added 15-crown-5 (0.21 mL, 1.06 mmol, 0.1 eq). The solution was cooled to 0° C. in an ice bath. After complete cooling of the solution, tetraethyl methylenebisphosphonate (3.46 g, 12.0 mmol, 1.1 eq) was added slowly via syringe and allowed to stir for 30 minutes to facilitate complete formation of the anion. Next, a solution of the crude bromide in THF was added slowly, via syringe, to the reaction vessel. The mixture was immediately removed from the ice bath and allowed to stir overnight. The solution was filtered through a bed of fluorasil and concentrated in vacuo. Purification was achieved using flash column chromatography (6% EtOH in hexanes) to afford the target bisphosphonate compound 16 in 47% (2.40 g) and compound 16b in 4% (0.75 g) yield over two steps. Compound 16, ¹H NMR δ 7.11 (1H, Jo=8.1, Jₒ=7.2 Hz), 7.04-7.00 (2H, m), 6.94 (1H, Jₒ=7.2 Hz), 5.25-5.16 (1H, m), 4.12-3.93 (8H, m), 3.23 (2H, d, J=8.1 Hz), 3.15 (2H, td, Jₜ=16.5, J_{d}=6.0 Hz), 2.69-2.47 (1H, m), 1.66 (3H, s), 1.64 (3H, s), 1.20 (12H, td, Jₜ=7.2, J_{d}=6.6 Hz) ¹³CNMR δ 141.5, 139.5 (1C, t, Jₚ=7.5 Hz), 132.1, 128.7, 128.0, 126.2, 126.0, 122.9, 62.4-62.1 (4C, m), 38.8 (1C, t, Jₚ=), 34.1, 30.9 (1C, t, Jₚ=5.1 Hz), 25.6, 17.6, 16.1 (4C, d, Jₚ=6.6 Hz) ³¹P NMR δ 23.6; dialkyl bisphosphonate compound 16b, ¹H NMR δ 7.28 (2H, d, Jₒ=6.9 Hz), 7.27 (2H), 7.15 (2H, t, Jₒ=8.1 Hz), 7.02 (2H, d, Jₒ=7.8 Hz), 5.35-5.29 (2H, m), 4.04-3.91 (8H, m), 3.37-3.26 (8H, m), 1.72 (6H, s), 1.70 (6H, s), 1.13 (12H, t, J₁=6.9 Hz), ¹³C NMR δ 140.5 (2C), 136.7 (2C), 132.0 (2C), 131.8 (2C), 129.0 (2C), 127.2 (2C), 126.4 (2C), 123.4 (2C), 62.0 (4C, t, Jₚ=3.4 Hz), 48.9 (1C, t, 4=137.8), 37.6 (2C, m), 34.3 (2C), 25.6 (2C), 17.7 (2C), 16.0 (4C, t, Jₚ=3.2 Hz). ³¹P NMR δ 25.0 HRMS (ESI, m/z) calcd for (M)⁺C₃₃H₅₀O₆P₂:604.3083. Found 604.3080.

### Example 3

### Synthesis of Compound 22

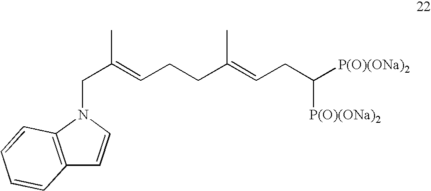

(3E,7E)-8-(1H-indol-1-yl)-4,8-dimethylnona-3,7-dien-1,1-bisphosphonic acid tetra sodium salt (22). A solution of 2,4,6-collidine (0.39 mL, 2.94 mmol) in CH₂Cl₂ was cooled to 0° C. in an ice bath and bromotrimethylsilane (0.38 mL, 2.94 mmol) was added. The solution was stirred for 20 minutes and compound 21 was added, via syringe, as a neat liquid. The solution was allowed to stir overnight and the volatiles were removed. Toluene was added, and the solvent was removed in vacuo. The resulting residue was treated with aqueous NaOH (1M, 1.9 mL, 1.9 mmol) and allowed to stir overnight. The mixture was poured into acetone, held at 3° C. for 72 hrs, and filtered. The retinate was dried, dissolved in H₂O, filtered and concentrated in vacuo to provide compound 22 (115 mg, 60%): ¹H NMR δ 7.60 (d, J=7.8 Hz, 1H), 7.39 (d, J=8.7 Hz, 1H), 7.21-7.12 (m, 2H), 7.06 (dd, J=7.5, 7.2 Hz, 1H), 6.47 (d, J=2.7 Hz, 1H), 5.57-5.46 (m, 1H), 5.36-5.30 (m, 1H), 4.57 (s, 2H), 2.51-2.37 (m, 2H), 2.16-1.93 (m, 4H), 1.62 (tt, J=21.6, 5.7 Hz, 1H), 1.57 (brs, 3H), 1.38 (brs, 3H); ¹³C NMR δ 136.3, 134.6, 131.8, 129.9, 128.5, 128.2, 127.5 (t, J=8.6 Hz, 1C), 121.7, 121.1, 119.7, 110.7, 100.3, 53.9, 41.7 (t, J=115.6, 1C), 39.1, 26.3, 26.2, 15.7, 13.3; ³¹P NMR δ 20.8.

Compound 21, was prepared as follows.

a) (2E,6E)-8-(1H-indol-1-yl)-3,7-dimethylocta-2,6-dienyl acetate (18). Indole (849 mg, 7.25 mmol) was dissolved in anhydrous DMF, the solution was cooled to 0° C. in an ice bath, and solid NaH (60% in oil, 320 mg, 8.00 mmol) was added cautiously. Once addition was complete, the solution was allowed to stir vigorously for 30 minutes. (2E,6E)-8-Bromo-3,7-dimethylocta-2,6-dienyl acetate (2.32 g, 8.43 mmol) was dissolved in THF and the resulting solution was added slowly to the reaction mixture via syringe. The mixture was removed from the ice bath and allowed to stir overnight. Water was added and the mixture was poured into ether. The solution was extracted with diethyl ether, and the extracts were dried (MgSO₄) and concentrated in vacuo. Final purification was achieved by flash column chromatography (10% EtOAc in hexanes) to afford compound 18 (1.10 g, 49%): ¹HNMR δ 7.62 (d, J=7.8 Hz, 1H), 7.32 (d, J=8.4 Hz, 2H), 7.22-7.14 (m, 1H), 7.11-7.04 (m, 1H), 6.49 (d, J=2.7 Hz, 1H), 5.35-5.25 (m, 2H), 4.56 (d, J=7.5 Hz, 2H), 4.48 (s, 2H), 2.21-2.12 (m, 2H), 2.10-2.02 (m, 2H), 2.05 (s, 3H), 1.67 (s, 3H), 1.51 (s, 3H); ¹³CNMR 171.1, 141.5, 136.3, 131.5, 128.5, 128.0, 126.9, 121.2, 120.7, 119.2, 118.7, 109.7, 101.0, 61.2, 54.1, 38.9, 25.7, 21.0, 16.3, 14.0; HRMS (EI⁺, m/z) calcd for C₂₀H₂₅NO₂: 311.1885. Found 311.1889.

b) (2E,6E)-8-(1H-indol-1-yl)-3,7-dimethylocta-2,6-diem-1-ol (19). Compound 18 (1.00 g, 3.22 mmol) was dissolved in MeOH, K₂CO₃ (2.5 g, 18.1 mmol) was added, and the solution was allowed to stir overnight. The solid K₂CO₃ was removed using gravity filtration and water was added. The solution was concentrated until ˜80% of the MeOH was removed, and then diethyl ether was added. The aqueous phase was extracted with diethyl ether, and the extracts were dried (MgSO₄) and concentrated in vacuo. Final purification was achieved by flash column chromatography (30% EtOAc in hexanes) to afford compound 19 (411 mg, 47%, 59% BRSM): ¹HNMR δ 7.62 (d, J=7.5 Hz, 1H), 7.32 (d, J=8.1 Hz, 1H), 7.20-7.05 (m, 3H), 6.49 (d, J=1.8 Hz, 1H), 5.35-5.23 (m, 2H), 4.59 (s, 2H), 4.09 (d, J=6.6 Hz, 2H), 2.18-2.11 (m, 2H), 2.07-2.01 (m, 2H), 1.63 (s, 3H), 1.53 (s, 3H); ¹³CNMR 138.3, 136.1, 131.2, 128.4, 128.1, 126.9, 123.8, 121.1, 120.7, 119.1, 109.7, 100.7, 59.0, 53.9, 38.8, 25.6, 16.0, 13.9; HRMS (EI⁺, m/z) calcd C₁₈H₂₃NO: 269.1780. Found 269.1770.

c) 1-((2E,6E)-8-Bromo-2,6-dimethylocta-2,6-dienyl)-1H-indole (20). A solution of compound 19 (1.00 g, 3.72 mmol) in THF was cooled to 0° C. in an ice bath. Triethylamine (0.67 mL, 4.81 mmol) was added followed by addition of MsCl (0.38 mL, 4.91 mmol). The resulting suspension was allowed to stir for 1 hr at 0° C., and solid LiBr (814 mg, 9.37 mmol) was added. The solution was allowed to warm to room temperature unassisted and stirred for 2 hrs. Water was added and the solution was extracted with diethyl ether. The combined organic extracts were dried over Na₂SO₄ and filtered through a bed of basic alumina. The solvent was removed in vacuo, and the resulting crude oil was used without additional purification in the following step (synthesis of compound 21).

d) (3E,7E)-8-(1H-indol-1-yl)-4,8-dimethylnona-3,7-dien-1,1-bisphosphonic acid tetraethyl ester (21). To a suspension of NaH (60% in oil, 150 mg, 3.75 mmol) in THF at 0° C. was added tetraethyl methylene bisphosphonate (1.10 g, 3.81 mmol) via syringe. The resulting mixture was allowed to stir for 20 minutes and compound 20 (1.24 g, 3.72 mmol) was added. The solution was allowed to warm to room temperature unassisted and stirred overnight. Water was added, the solution was extracted with diethyl ether, and the combined extracts were dried (MgSO₄) and concentrated in vacuo. Final purification was achieved by flash column chromatography (8 EtOH in hexanes) to afford desired bisphosphonate compound 21 (625 mg, 31%): ¹HNMR δ 7.61 (d, J=7.5 Hz, 1H), 7.33 (d, J=8.4 Hz, 1H), 7.18 (t, J=7.2 Hz, 1H), 7.11-7.05 (m, 2H), 6.49 (d, J=2.4 Hz, 1H), 5.35-5.31 (m, 2H), 4.59 (s, 2H), 4.21-4.13 (m, 8H), 2.73-2.54 (m, 2H), 2.32 (tt, J=23.4, 6.3 Hz, 1H), 2.18-2.09 (m, 2H), 2.05-1.94 (m, 2H), 1.63 (s, 3H), 1.50 (s, 3H), 1.33 (t, J=6.9 Hz, 12H); ¹³CNMR 136.2, 136.1, 131.0, 128.5, 128.0, 127.5, 122.1 (t, J=7.3 Hz, 1C), 121.2, 120.6, 119.1, 109.6, 100.9, 62.3 (dd, J=8.5, 7.0 Hz, 4C), 54.1, 39.1, 37.3 (t, J=131.8, 1C), 26.1, 23.9 (t, J=4.8 Hz, 1C), 16.3 (d, J=7.4 Hz, 4C), 15.9, 13.9; HRMS (EI⁺, m/z), calcd C₂₇H₄₃NO₆P₂: 539.2566. Found 539.2567.

### Example 4

The following illustrate representative pharmaceutical dosage forms, containing a compound of formula I (‘Compound X’), for therapeutic and/or prophylactic use in humans.

| (i) Tablet 1 | mg/tablet |
|---|---|
| Compound X = | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X = | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X = | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |

| (vi) Aerosol | mg/can |
|---|---|
| Compound X = | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents cited herein are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A therapeutic method to treat a mycobacterium infection in a mammal comprising administering to the mammal a compound of formula I: 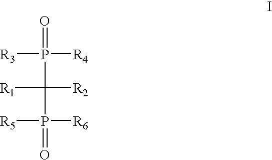
wherein:
R₁ is a saturated or unsaturated (C₅-C₂₀)alkyl chain that optionally comprises one or more aryl or heteroaryl rings in the chain wherein (C₅-C₂₀)alkyl is optionally substituted with one or more halo cyano, nitro, carboxy, trifluoromethyl, trifluoromethoxy, NRₘRₙ, or S(O)₂NRₚR_{q} and wherein any aryl or heteroaryl is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d};
R₂ is H, halo, OH, trifluoromethyl, —ORₑ, NR_{f}R_{g} or a saturated or unsaturated (C₁-C₆)alkyl wherein (C₁-C₆)alkyl is optionally substituted with one or more halo;
each R₃, R₄, R₅, and R₆ is independently OH or (C₁-C₆)alkoxy;
each Rₐ and R_{b} is independently H, (C₁-C₆)alkyl, or aryl; or Rₐ and R_{b} together with the nitrogen to which they are attached form a pyrrolidino, piperidino, morpholino, or thiomorpholino ring;
each R_{c} and R_{d} is independently H, (C₁-C₆)alkyl, or aryl; or R_{c} and R_{d} together with the nitrogen to which they are attached form a pyrrolidino, piperidino, morpholino, or thiomorpholino ring;
each Rₑ is independently (C₁-C₆)alkyl or aryl;
each R_{f} and R_{g} is independently H, (C₁-C₆)alkyl, or aryl; or R_{f} and R_{g} together with the nitrogen to which they are attached form a pyrrolidino, piperidino, morpholino, or thiomorpholino ring;
each Rₘ and Rₙ is independently H, (C₁-C₆)alkyl, or aryl; or Rₘ and Rₙ together with the nitrogen to which they are attached form a pyrrolidino, piperidino, morpholino, or thiomorpholino ring;
each Rₚ and R_{q} is independently H, (C₁-C₆)alkyl, or aryl; or Rₚ and R_{q} together with the nitrogen to which they are attached form a pyrrolidino, piperidino, morpholino, or thiomorpholino ring; and
wherein any aryl of Rₐ, R_{b}, R_{c}, R_{d}, Rₑ, R_{f}, R_{g}, Rₘ, Rₙ, Rₚ or R_{q} is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₛRₜ, or S(O)₂NRₛRₜ wherein each Rₛ and Rₜ is independently H or (C₁-C₆)alkyl;
or a pharmaceutically acceptable salt or prodrug thereof.

2. The method of claim 1 wherein:
R₁ is a saturated or unsaturated (C₅-C₂₀)alkyl chain that optionally comprises one or more aryl or heteroaryl rings in the chain wherein (C₅-C₂₀)alkyl is optionally substituted with one or more halo cyano, nitro, carboxy, trifluoromethyl, trifluoromethoxy, NRₘRₙ, or S(O)₂NRₚR_{q} and wherein any aryl or heteroaryl is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d};
R₂ is H, halo, OH, trifluoromethyl, —ORₑ, NR_{f}R_{g} or a saturated or unsaturated (C₁-C₆)alkyl wherein (C₁-C₆)alkyl is optionally substituted with one or more halo;
each R₃, R₄, R₅, and R₆ is independently OH or (C₁-C₆)alkoxy;
each Rₐ and R_{b} is independently H, (C₁-C₆)alkyl, or aryl; or Rₐ and R_{b} together with the nitrogen to which they are attached form a pyrrolidino, piperidino, morpholino, or thiomorpholino ring;
each R_{c} and R_{d} is independently H, (C₁-C₆)alkyl, or aryl; or R_{c} and R_{d} together with the nitrogen to which they are attached form a pyrrolidino, piperidino, morpholino, or thiomorpholino ring;
each Rₑ is independently (C₁-C₆)alkyl or aryl;
each R_{f} and R_{g} is independently H, (C₁-C₆)alkyl, or aryl; or R_{f} and R_{g} together with the nitrogen to which they are attached form a pyrrolidino, piperidino, morpholino, or thiomorpholino ring;
each Rₘ and Rₙ is independently H, (C₁-C₆)alkyl, or aryl; or Rₘ and Rₙ together with the nitrogen to which they are attached form a pyrrolidino, piperidino, morpholino, or thiomorpholino ring; and
each Rₚ and R_{q} is independently H, (C₁-C₆)alkyl, or aryl; or Rₚ and R_{q} together with the nitrogen to which they are attached form a pyrrolidino, piperidino, morpholino, or thiomorpholino ring;
or a pharmaceutically acceptable salt or prodrug thereof to the mammal.

3. The method of claim 1 wherein the mycobacterium infection is tuberculosis.

4. A method for inhibiting the activity of a mycobacterial polyprenyl pyrophosphate synthase comprising contacting the mycobacterial polyprenyl pyrophosphate synthase with an effective amount of a compound of formula I as described in claim 1 or a pharmaceutically acceptable salt or prodrug thereof.

5. The method of claim 1 wherein R₁ is an unsaturated (C₅-C₂₀)alkyl chain.

6. The method of claim 1 wherein R₁ is a saturated or unsaturated (C₅-C₂₀)alkyl chain that comprises one or more aryl rings in the chain.

7. The method of claim 1 wherein R₁ is of the formula, 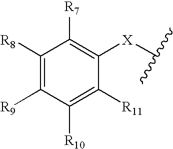
wherein:
one of R₇, R₈, R₉, R₁₀ and R₁₁ is Y and the others are each independently H, halo, cyano, nitro, carboxy, trifluoromethyl, trifluoromethoxy, (C₁-C₆)alkyl, NRⱼRₖ, or S(O)₂NRⱼRₖ wherein Rj and Rₖ are each H or (C₁-C₆)alkyl;
Y is a saturated or unsaturated (C₁-C₂₀)alkyl; and
X is (CRₕRᵢ)ₙ wherein n is 0, 1, 2, 3, 4, or 5 and for each CRₕRᵢ; Rₕ and Rᵢ are each independently H or (C₁-C₃)alkyl;
provided that the sum of the carbons of X and Y is 5 to 20.

8. The method of claim 7 wherein Rₕ and Rᵢ are each H.

9. The method of claim 7 wherein n is 1.

10. The method of claim 7 wherein R₈ is Y.

11. The method of claim 7 wherein R₉ is Y.

12. The method of claim 7 wherein Y is a saturated or unsaturated (C₅-C₂₀)alkyl.

13. The method of claim 7 wherein Y is 3-methyl-2-buten-1-yl.

14. The method of claim 1 wherein R₁ is

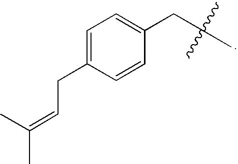

15. The method of claim 1 wherein R₁ is

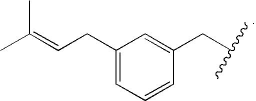

16. The method of claim 1 wherein R₁ is a unsaturated (C₅-C₂₀)alkyl chain that comprises a heteroaryl ring in the chain.

17. The method of claim 16 wherein the heteroaryl ring is indolyl.

18. The method of claim 1 wherein R₁ is

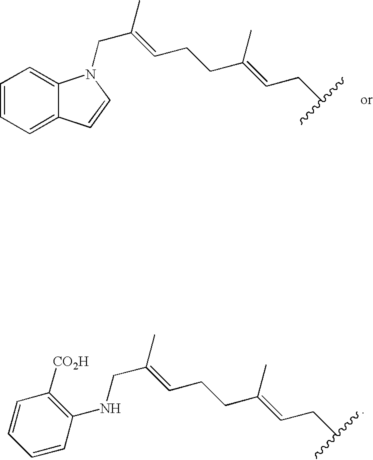

19. The method of any one of claim 1 wherein R₂ is saturated or unsaturated (C₁-C₆)alkyl, OH or H.

20. The method of claim 1 wherein R₃, R₄, R₅ and R₆ are each OH.

21. The method of claim 1 wherein the compound of formula I is: 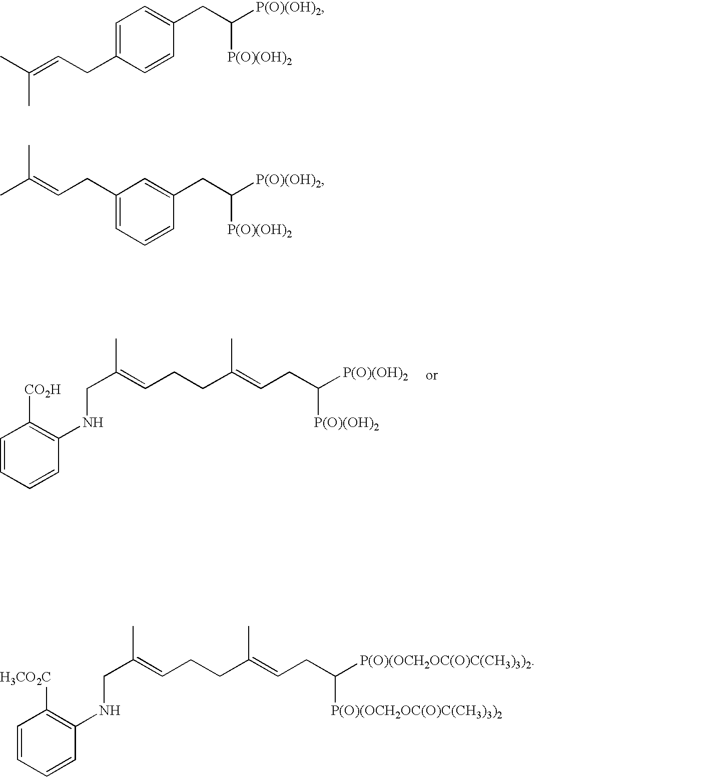
or a pharmaceutically acceptable salt or prodrug thereof.

22. A compound of formula I: 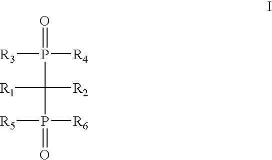
wherein:
R₁ is a saturated or unsaturated (C₅-C₂₀)alkyl chain that comprises one or more heteroaryl rings and optionally comprises one or more aryl rings in the chain wherein (C₅-C₂₀)alkyl is optionally substituted with one or more halo, cyano, nitro, carboxy, trifluoromethyl, trifluoromethoxy, NRₘRₙ, or S(O)₂NRₚR_{q} and wherein any aryl or heteroaryl is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₐR_{b}, or S(O)₂NR_{c}R_{d};
provided that when R₁ is a saturated or unsaturated (C₅-C₂₀)alkyl chain that comprises one pyridine ring, the pyridine ring is not linked to the alkyl chain of R₁ through the pyridine nitrogen;
R₂ is H, halo, OH, trifluoromethyl, —ORₑ, NR_{f}R_{g} or a saturated or unsaturated (C₁-C₆)alkyl wherein (C₁-C₆)alkyl is optionally substituted with one or more halo;
each R₃, R₄, R₅, and R₆ is independently OH or (C₁-C₆)alkoxy;
each Rₐ and R_{b} is independently H, (C₁-C₆)alkyl, or aryl; or Rₐ and R_{b} together with the nitrogen to which they are attached form a pyrrolidino, piperidino, morpholino, or thiomorpholino ring;
each R_{c} and R_{d} is independently H, (C₁-C₆)alkyl, or aryl; or R_{c} and R_{d} together with the nitrogen to which they are attached form a pyrrolidino, piperidino, morpholino, or thiomorpholino ring;
each Rₑ is independently (C₁-C₆)alkyl or aryl;
each R_{f} and R_{g} is independently H, (C₁-C₆)alkyl, or aryl; or R_{f} and R_{g} together with the nitrogen to which they are attached form a pyrrolidino, piperidino, morpholino, or thiomorpholino ring;
each Rₘ and Rₙ is independently H, (C₁-C₆)alkyl, or aryl; or Rₘ, and Rₙ together with the nitrogen to which they are attached form a pyrrolidino, piperidino, morpholino, or thiomorpholino ring;
each Rₚ and R_{q} is independently H, (C₁-C₆)alkyl, or aryl; or Rₚ and R_{q} together with the nitrogen to which they are attached form a pyrrolidino, piperidino, morpholino, or thiomorpholino ring; and
wherein any aryl of Rₐ, R_{b}, R_{c}, R_{d}, Rₑ, R_{f}, R_{g}, Rₘ, Rₙ, Rₚ or R_{q} is optionally substituted with one or more (C₁-C₆)alkyl, (C₁-C₆)alkoxy, (C₁-C₆)alkanoyl, (C₁-C₆)alkanoyloxy, (C₁-C₆)alkoxycarbonyl, halo, cyano, nitro, carboxy, trifluoromethyl, trifluoromethoxy, NRₛRₜ, or S(O)₂NRₛRₜ wherein each Rₛ and Rₜ is independently H or (C₁-C₆)alkyl;
or a salt or prodrug thereof;
provided that the compound is not

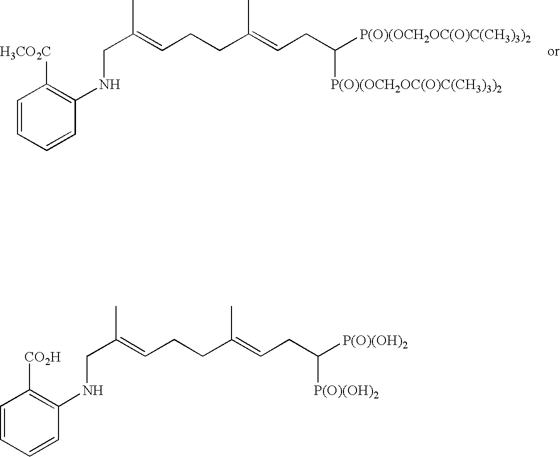

23. The compound 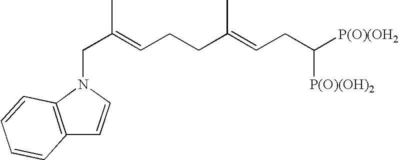
or a salt or prodrug thereof.

24. A pharmaceutical composition comprising a compound of formula I as described in claim 22, or a pharmaceutically acceptable salt or prodrug thereof, and a pharmaceutically acceptable diluent or carrier.

25. A therapeutic method to treat a mycobacterium infection in a mammal comprising administering to the mammal a compound of formula I as described in claim 22 or a pharmaceutically acceptable salt or prodrug thereof.

26. The method of claim 25 wherein the mycobacterium infection is tuberculosis.

27. A method for inhibiting the activity of a mycobacterial polyprenyl pyrophosphate synthase comprising contacting the mycobacterial polyprenyl pyrophosphate synthase with an effective amount of a compound of formula I as described in claim 22 or a pharmaceutically acceptable salt or prodrug thereof.

28. The method of claim 1 wherein the compound is a prodrug of the compound of formula I.

29. The method of claim 28 wherein one or more of R₃, R₄, R₅ or R₆ is —OCH₂OC(O)C(CH₃)₃).
